(19) United States
(12) Patent Application Publication
(10) Pub. No.: US 2008/0027372 A1
(43) Pub. Date: Jan. 31, 2008

(54) Medication port for a ventilation machine

(76) Inventor: Stanley Baldwin, Riverside, CA (US)

Correspondence Address: MICHAEL A. SHIPPEY, PH. D., LAW OFFICES OF KARLA SHIPPEY, 4848 LAKEVIEW AVENUE, SUITE E, YORBA LINDA, CA, 92886, US

(21) Appl. No.: 11/497,111

(22) Filed: Jul. 31, 2006

(51) Int. Cl. A61M 37/00

(52) U.S. Cl. 604/23; 604/24

(57) ABSTRACT

The current invention is a port for the introduction of a nebulizer or other injector of medication to the lungs of a patient. It allows for the administering of medication without the need to remove the patient from a ventilator. This in turn eliminates certain medical complications that often arise when a patient is removed from a ventilator, even briefly.

### BACKGROUND OF THE INVENTION

There have been rapid rates of invention in the area of medical devices in the past decade. This is especially true for medical devices for assisted breathing, more commonly known as ventilators.

Ventilators are used to assist the breathing of individuals who have difficulty breathing on their own. This happens in certain surgical procedures, but more often to people with respiratory illnesses such as emphysema. Prime candidates for a ventilator are premature babies, who often do not have fully developed lungs at birth.

Nebulizers are simple devices that are often used with ventilators to deliver medications to patients. A nebulizer, charged with a medicant (usually a liquid medicine), will create an aerosol, either directly or with the aid of pressurized gas. The gas, typically air or a component thereof, thus loaded with the medicine in the form of an aerosol, is then delivered to the patient via the ventilator. Thus is a sick patient on a ventilator often given medication.

There have been many patents and patent applications, not surprisingly, in this field. R. A. Niles et. al, in U.S. Pat. No. 7,036,500 B2, which issued on May 2, 2006, reveal a nebulizer with a supplemental gas inlet port. Further improvements to this same invention are found in published patent application of Ser. No. 10/842,334, filed on May 10, 2004 by the same inventors.

T. M. Mendenhall, in U.S. Pat. No. 6,772,754 B1, which issued on Aug. 10, 2004, describes a nebulizer with a breath-actuated controller.

A group of inventors led by Fink have filed a number of published patent applications in this field: Ser. No. 10/828,765, filed on Apr. 20, 2004, first inventor Fink, on an aerosol delivery system; Ser. No. 10/883,115, filed on Jun. 30, 2004, first inventor Ivri, on an improved aerosol delivery system; and published patent application of Ser. No. 11/080,279, filed on Mar. 14, 2005 by first inventor Fink, on a method and treatment for lung surfactant dysfunction.

The same group, J. B. Fink, et. al, in U.S. patent application Ser. No. 11/090,328, published on Oct. 6, 2005, describe a method of treating a patient involving taking the patient off the breathing device prior to administering medication via a nebulizer.

The last named invention specifies that the ventilator is to be switched off, or the patient taken off of the ventilator, in order to introduce the aerosol medication by nebulizer. Most of the prior art seems to follow the same practice. For many patients, this practice works well.

In order to introduce medications through a ventilator port of a high frequency ventilator, the cap to the port must be removed to insert a nebulizer. When this standard practice is done the high frequency ventilator loses pressure and stops ventilation to the patient.

However, many populations of patients, many whom are premature infants, suffer from the loss of pressure even for a very brief period of time. This current above practice for the introduction of aerosolized medications with the concurrent pressure loss can have deleterious effects to the patient's lungs. The end result is the mean airway pressure drops resulting in de-recruitment of the patient alveoli (collapse).

Repeated administration of medications over time causes a loss of functional residual capacity of the lung which the ventilator mean pressure is designed to increase and improve breathing function of the patient. To reverse this loss of function the operator will many times have to increase pressure over time just to get the patient back to their baseline values. This can cause further damage to the patient's lung and require increasing the time of mean mechanical ventilation and stay in an intensive care unit.

What is needed is a means to introduce medication, particularly as an aerosol, while the ventilator is functioning, and keeping the patient's alveoli full and un-collapsed. This is the problem that the current invention is designed to solve.

### SUMMARY OF THE CURRENT INVENTION

The current invention is a port for the introduction of a nebulizer or other injector of medication to the lungs of a patient who is breathing with the aid of a medical assisted-breathing device, commonly termed a ventilator. The invention allows for the administering of medication without the need to remove the patient from the ventilator. This in turn eliminates certain complications that often arise when a patient is removed from a ventilator, even briefly.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is an illustration of the current device in use, attached to the inhalation line of a ventilator.

FIG. 2 is an enlarged perspective view of the current device, showing details of its attachment to the inhalation line of a ventilator.

FIG. 3 is a further enlarged perspective view of the detail of the current invention, with a nebulizer being detached from the current invention.

FIG. 4 shows a view of the current invention from the side, with details of the one-way valve of the current invention displayed. The nebulizer is still detached from the device.

FIG. 5 illustrates the attachment of a nebulizer to the device of the current invention.

FIG. 6 shows the device of the current invention from a top plan view.

FIG. 7 shows the device of the current invention from a bottom plan view.

### DESCRIPTION OF THE CURRENT INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as representative basis for teaching one skilled in the art to employ the present invention in virtually any appropriately detailed system, structure or manner.

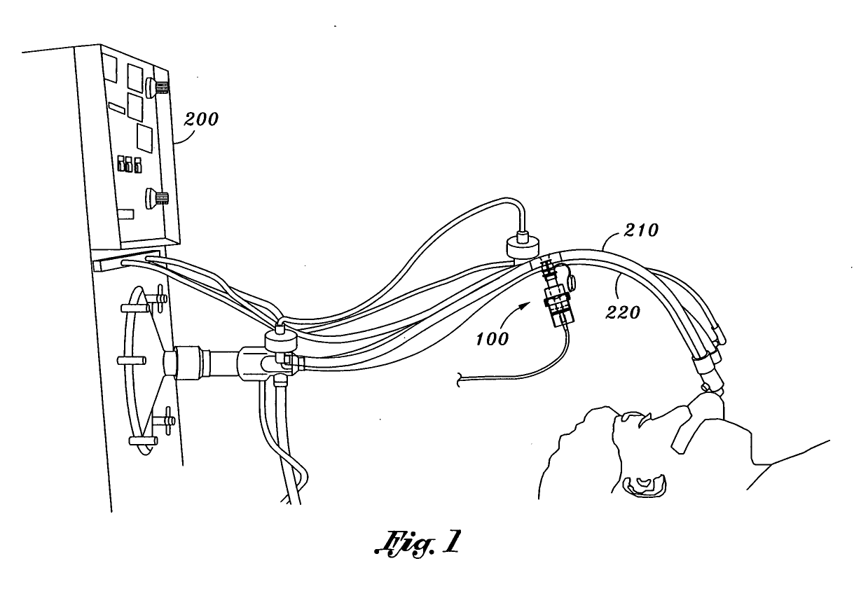

FIG. 1 displays a patient breathing on a modern high frequency ventilator. The ventilator itself 200, which is not part of the current invention, is shown at the left side of the figure. The ventilator is comprised of an upper electronics controller box, a lower box containing a diaphragm oscillator, and a plurality of lines 210, 220 for carrying inhalation air or oxygen, and exhalation gases. The gas lines are typically made of flexible clear plastic tubing.

A standard set of lines will include at least an inhalation line 210 and an exhalation line 220. A standard port may be placed in the inhalation line, approximately at the location of the current invention 100, displayed in FIG. 1. The standard port is no more than a 4 mm ID circular opening in the line, with a cap to seal the aperture when the ventilator is active, to prevent gases from escaping. In order to introduce medications through the standard port, the ventilator must be turned off, or the inhalation line removed from the ventilator by a closed switch or other means. The medication can then be introduced, and the cap replaced, followed by the ventilator switched on or reconnected to the gas line.

However, a group of patients, many of whom are premature babies, suffer from being removed from the ventilator, even for a very brief period of time. With current above practice, the oscillator loses pressure, the patient's mean airway pressure drops, resulting in de-recruitment of alveoli (collapsing) in the lung. It takes a long time to re-expand the tiny alveoli within the lung.

What is needed is a method to introduce medication without removing the patient from the ventilator, even briefly. This is the problem solved by the device of the current invention.

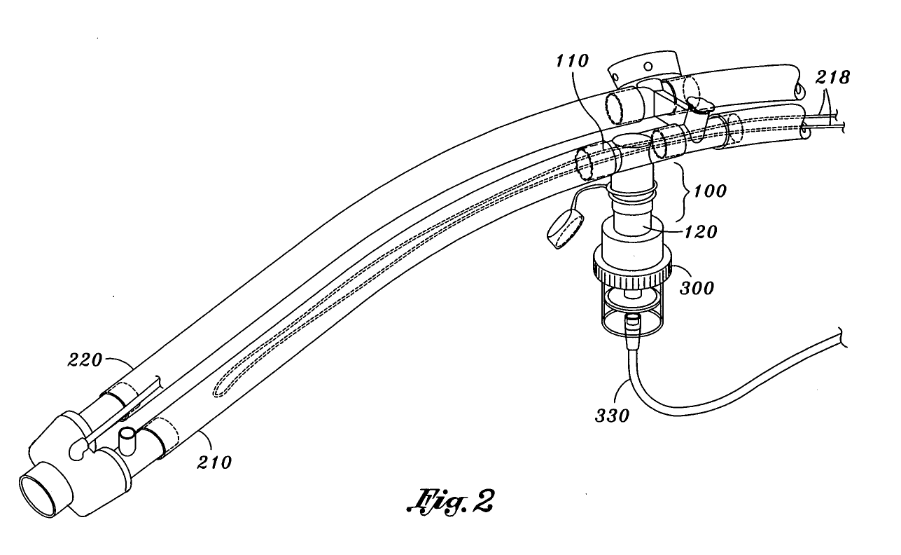

FIG. 2 shows the current invention 100 in place in the inhalation line 210 of a ventilator. Into the inhalation line 210 is installed the device 100 of the current invention. Horizontal arm 110 of the current invention fits firmly inside inhalation line 210. The horizontal arm 110 is hollow, enabling gases and wires, such as heating wires 218, to pass through. The invention 100 can thus be in place and in used without interfering with the operation of the ventilator.

Also in FIG. 2 is seen a nebulizer device 300 attached to the open end of the vertical arm 120 of the current invention. Nebulizer 300 is not part of the current invention. However, a gas operated nebulizer is a common method for introduction of medication as an aerosol into gas being inhaled by a patient, and thereby into patient's lungs. The aerosol is created by passing pressurized gas from a gas line 330 into the liquid medication within nebulizer 300. The gas further provides impetus for the aerosol to move through port 100 into line 210, and thence to the patient.

The purpose of the current invention is to allow the aerosol generated by the nebulizer to enter the inhalation line of a ventilator during operation, without any inhibition of the operation.

FIG. 3 shows the current invention after the nebulizer 300 has been detached. Normally, after detachment of a nebulizer or other device utilizing the port 100, the port will be closed off by cap 140. Cap 140 remains attached to device 100 by a retainer cord 140. However, whether the cap 140 remains attached or not, the port still seals off the inhalation line from the outside, and allows the ventilator to keep intact and operational.

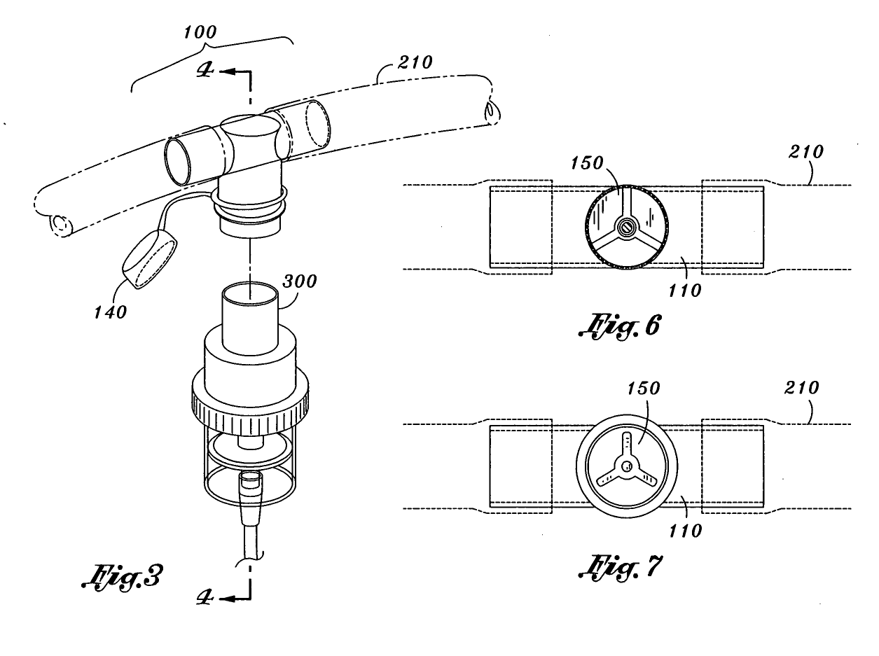

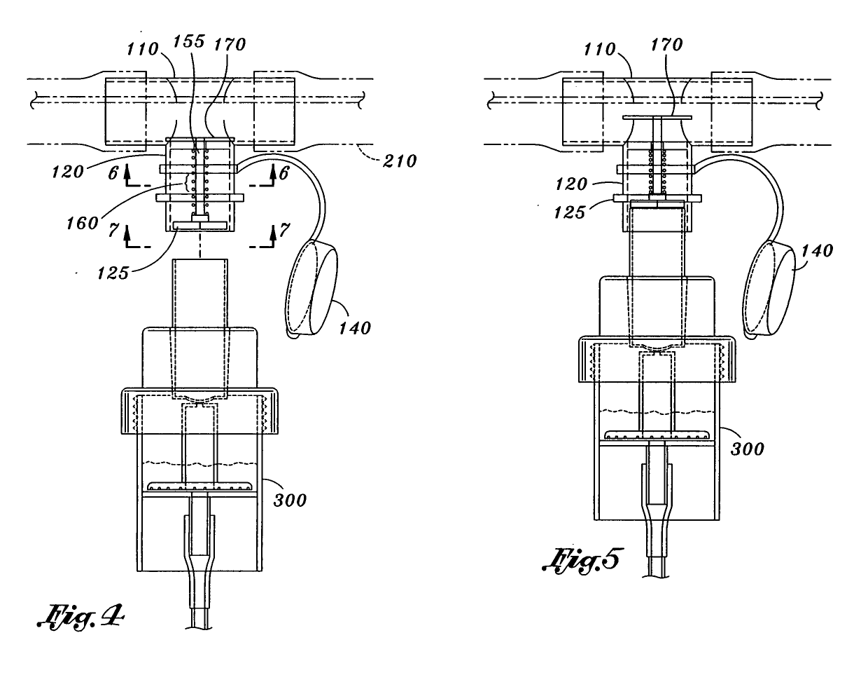

The current invention is seen to advantage in FIG. 4. The active part of the current invention is the one-way valve 150. Housed within vertical arm 120, the valve has a spring 160 which, when arm 120 is free at its lower end, forces top plate 170 against the top of vertical arm 120, thus sealing the aperture connecting vertical arm 120 to horizontal arm 110. This in turn preserves the integrity of the line into which the device is inserted, in this case, inhalation line 210. Cap 140, when affixed, is backup to the seal provided by top plate 170.

When an appropriately sized tube, such as the inlet nozzle of nebulizer 300, is inserted into the free end of vertical arm 120, lower plate 125 is pushed upwards, pushing shaft 155 upwards, and ultimately forcing plate 170 up into the central passageway of gas inhalation line 210. As a result, plate 170 no longer seals off vertical arm 120. Gases can therefore move from nebulizer 300 through vertical arm 120, into the central passageway of gas inhalation line 210, and on to the patient. This is clearly seen in the portrayal in FIG. 5.

It will be seen from the figures that the current invention can be an external unit that is added to an existing gas inhalation line on a ventilator. The line 210 is cut, the heating wires removed, the horizontal arm 110 of the current invention placed into the cut, and the cut ends of the line affixed to either end of the horizontal arm. The heating wires are then replaced, threaded through the gas line and horizontal arm.

It is of course possible to manufacture a new gas line with T-structure as an integral element thereof. In that case, one needs to insert a one-way valve into vertical arm 120 to attain the current invention.

While the invention has been described in connection with a preferred embodiment or embodiments, it is not intended to limit the scope of the invention to the particular form set forth, but on the contrary, it is intended to cover such alternatives, modifications, and equivalents as may be included within the spirit and scope of the invention as defined by the appended claims.

I claim:

1. In a medical device comprising at least one line for transmitting gases, a port, said port comprising
a. A connection to said gas line;
b. A one-way valve permitting transmission of materials into said gas line, but preventing materials from escaping from said gas line;
c. Means for connecting an injector device; wherein,
d. All connections and valves are gas tight to prevent leakage of gas.

2. The device of claim 1, further comprising a cap to prevent leakage of gas when said port is not in use.

3. The device of claim 1, wherein said injector device comprises a nebulizer.

4. The device of claim 1, wherein said device comprises an apparatus to assist breathing of a patient.

5. The device of claim 4, said device further comprising an inlet line for delivering oxygen to the patient, wherein said port is located along said inlet line.

6. In a medical device comprising a plurality of lines for transmitting breathing gases, a port, said port comprising
a. A connection to said gas line;
b. A one-way valve permitting transmission of materials into said gas line, but preventing materials from escaping from said gas line;
c. A cap, for use to seal the port when said port is not in use; and,
d. Means for connecting an injector device to permit injection of materials into said gas line via said valve; wherein,
e. All connections and valves are gas tight to prevent leakage of gas.

7. An external port, for the addition to a medical device comprising at least one line for transmitting gases, said port comprising:
a. A hollow horizontal arm for insertion into said gas line, said horizontal arm permitting the passage of gas therethrough;
b. A hollow vertical arm, perpendicular to said horizontal arm, and connected thereto;
c. A one-way valve permitting transmission of materials into said gas line, but preventing materials from escaping from said gas line;
d. Said valve being disposed within said vertical arm; and,
e. Means for connecting an injector device to the end of said vertical arm; wherein,
f. All connections, arms, and valves are gas tight to prevent leakage of gas.
